(19) United States
(12) Patent Application Publication
(10) Pub. No.: US 2009/0118604 A1
(43) Pub. Date: May 7, 2009

(54) ANALYTE MONITORING SYSTEM HAVING BACK-UP POWER SOURCE FOR USE IN EITHER TRANSPORT OF THE SYSTEM OR PRIMARY POWER LOSS

(75) Inventors: Luong Ngoc Phan, San Clemente, CA (US); Michael J. Higgins, Huntington Beach, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/263,151

(22) Filed: Oct. 31, 2008

Related U.S. Application Data
(60) Provisional application No. 60/985,112, filed on Nov. 2, 2007.

(51) Int. Cl. A61B 5/1477; A61B 5/1468

(52) U.S. Cl. 600/345

(57) ABSTRACT

An analyte monitoring system includes a biosensor for detecting an analyte concentration in blood. The monitoring system includes first and second power sources, each selectively couplable to the biosensor for providing power to the biosensors. A sensor may be associated with the first power source and senses the output thereof. A selector is coupled to both the first and second power sources and the biosensor, such that it may selectively couple an output or outputs of either the first or second power sources to the biosensor. In operation, the first power source is coupled to the biosensor to thereby bias the sensor. If the sensor indicates that the first power source is not providing power to the biosensor, the selector decouples the first power source from the biosensor and couples the second power source to the biosensor to thereby maintain the biosensor in a biased state.

### CROSS-REFERENCE TO RELATED APPLICATIONS

This application claims priority from U.S. provisional patent application No. 60/985,112, filed on Nov. 2, 2007, which is also hereby incorporated herein by reference.

### BACKGROUND

1. Field of the Invention

The invention relates generally to an analyte monitoring system. More specifically, the invention relates to an electronic system for providing backup bias power for an electro-chemical biosensor, such as an amperometric, potentiometric, or similar type biosensor, that requires voltage biasing for operation.

2. Description of Related Art

Controlling blood glucose levels for diabetics and other patients can be a vital component in critical care, particularly in an intensive care unit (ICU), operating room (OR), or emergency room (ER) setting where time and accuracy are essential. Presently, the most reliable way to obtain a highly accurate blood glucose measurement from a patient is by a direct time-point method, which is an invasive method that involves drawing a blood sample and sending it off for laboratory analysis. This is a time-consuming method that is often incapable of producing needed results in a timely manner. Other minimally invasive methods such as subcutaneous methods involve the use of a lancet or pin to pierce the skin to obtain a small sample of blood, which is then smeared on a test strip and analyzed by a glucose meter. While these minimally invasive methods may be effective in determining trends in blood glucose concentration, they do not track glucose accurately enough to be used for intensive insulin therapy, for example, where inaccuracy at conditions of hypoglycemia could pose a very high risk to the patient.

Electro-chemical biosensors have been developed for measuring various analytes in a substance, such as glucose. An analyte is a substance or chemical constituent that is determined in an analytical procedure, such as a titration. For instance, in an immunoassay, the analyte may be the ligand or the binder, where in blood glucose testing, the analyte is glucose. Electro-chemical biosensors comprise eletrolytic cells including electrodes used to measure an analyte. Two types of electro-chemical biosensors are potentiometric and amperometric biosensors.

Amperometric biosensors, for example, are known in the medical industry for analyzing blood chemistry. These types of sensors contain enzyme electrodes, which typically include an oxidase enzyme, such as glucose oxidase, that is immobilized behind a membrane on the surface of an electrode. In the presence of blood, the membrane selectively passes an analyte of interest, e.g. glucose, to the oxidase enzyme where it undergoes oxidation or reduction, e.g. the reduction of oxygen to hydrogen peroxide. Amperometric biosensors function by producing an electric current when a potential sufficient to sustain the reaction is applied between two electrodes in the presence of the reactants. For example, in the reaction of glucose and glucose oxidase, the hydrogen peroxide reaction product may be subsequently oxidized by electron transfer to an electrode. The resulting flow of electrical current in the electrode is indicative of the concentration of the analyte of interest.

FIG. 1 is a schematic diagram of an exemplary electro-chemical biosensor, and specifically a basic amperometric biosensor 10. The biosensor comprises two working electrodes: a first working electrode 12 and a second working electrode 14. The first working electrode 12 is typically an enzyme electrode either containing or immobilizing an enzyme layer. The second working electrode 14 is typically identical in all respects to the first working electrode 12, except that it may not contain an enzyme layer. The biosensor also includes a reference electrode 16 and a counter electrode 18. The reference electrode 16 establishes a fixed potential from which the potential of the counter electrode 18 and the working electrodes 12 and 14 are established. In order for the reference electrode 16 to function properly, no current must flow through it. The counter electrode 18 is used to conduct current in or out of the biosensor so as to balance the current generated by the working electrodes. The four electrodes together are typically referred to as a cell. During operation, outputs from the working electrodes are monitored to determine the amount of an analyte of interest that is in the blood, Potentiometric biosensors operate in a similar manner to detect the amount of an analyte in a substance.

While electro-chemical biosensors containing eletrolytic cells, such as amperometric and potentiometric biosensors, are a marked improvement over more conventional analyte testing devices and methods, there are some potential drawbacks to their use. For example, electro-chemical biosensors typically require time for chemistry cell alignment after initial biasing and prior to calibration and use. The process beginning from a time when the bias signals are applied until the cell is in full alignment (i.e., steady state) can be anywhere from a few minutes to more than an hour (e.g., 15 minutes to 1.5 hours). The time for chemistry cell alignment is typically referred to as run-in time.

Significant delays in run-in time can be problematic, especially where the biosensor is in use and there is an unexpected loss of power to the cell. For example, if the electronics to the biosensor is unplugged during the transport of the patient or to reconfigure the various electric lines, IVs, tubes, etc. connected to a patient, the biometric sensor will experience disruption of steady state that may require significant time for the biosensor to again be operational. This may be a particular problem where the patient is entering surgery, where blood content monitoring is critical.

In light of the above, systems and methods are needed to monitor loss of power to electro-chemical biosensors having eletrolytic cells and provide auxiliary power for maintaining cell alignment during the power outage or disconnect.

### BRIEF SUMMARY OF THE INVENTION

The present invention provides systems and methods for maintaining cell alignment of an electro-chemical biosensor having an eletrolytic cells during transport or power outage. The systems and methods of the present invention provide a second or auxiliary power source for providing bias power to the biosensor. A sensor is associated with the system for detecting when there has been or will be a loss of bias power from the primary power source. In which instance, the second or auxiliary power source is coupled to the biosensor so as to maintain bias within the cell. As such, the systems and methods of the present invention significantly reduce and/or alleviate run-in time delays associated with the biosensor.

According to one embodiment of the present invention, an analyte monitoring system is provided that comprises a biosensor capable of sensing an analyte concentration and outputting a signal corresponding to the analyte concentration. Associated with the biosensor are first and second power sources, each selectively couplable to the biosensor for providing power thereto. A selector is coupled to the first and second power sources and selectively couples one of the first and second power sources to the biosensor.

In some embodiments, the system includes a sensor capable of sensing operation of the first power source. In this embodiment, the selector selectively couples one of the first and second power sources to the biosensor based on an output of the sensor.

In some embodiments, the sensor is either a current or a voltage sensor, which is in electrical communication with an output of the first power source. In operation, if the sensor indicates that the first power source is not outputting a current or voltage, the selector couples the second power source to the biosensor.

The present invention does not require that the sensor monitor the output of the power supply. Instead, the selector could be a switch that is accessible by an operator. In this embodiment, the operator could alter the position of the switch to indicate that the first power source is either disabled or soon to be disabled.

In other embodiments, the first power source may include a power down mode, and the sensor could be associated with the power source and sense that the power source is powering down.

There are various alternatives configurations for the selector. The selector may be a switch having contacts electrically coupled respectively to the first and second power sources, wherein the switch is capable of selectively coupling either of the first or second power sources to the biosensor. The switch could either be or be associated with an electronic device such as an ASIC or microprocessor that monitors the sensor and selectively connects either of the first or second power sources to the biosensor.

In some embodiments, the electro-chemical biosensor may comprise two or more electrodes. The second power source is capable of providing either one or different bias signals to the electrodes, based on the requirement of each electrode for maintaining cell alignment.

In one embodiment of the present invention, the analyte monitoring system may include an electro-chemical biosensor comprising at least a reference electrode and a work electrode. The system may further include a potentiostat as a first power source. In this embodiment, the second power source or auxiliary power source is configured so as to provide a bias signal to both the reference and work electrodes. When the sensor indicates that the potentiostat is not supplying power to the biosensor, the selector connects the second or auxiliary power source to the reference and work electrodes of the biosensor. In other embodiments, the first power source could be an amperostat, sometimes referred to as a galvanostat.

The present invention also provides methods for controlling operation of an electro-chemical biosensor. For example, in one embodiment, the method may comprise providing an electro-chemical biosensor capable of sensing an analyte concentration and outputting a signal corresponding to the analyte concentration. The method selectively couples either a first or a second power source to the biosensor based on whether one of the power sources is supplying power, so as to maintain the biosensor in a biased state. For example, the method couples the first power source to the biosensor if the first power source is outputting a signal to the sensor and couples the second power source to the biosensor if the first power is not outputting a signal to the sensor.

### BRIEF DESCRIPTION OF THE DRAWINGS

Henceforth reference is made the accompanied drawings and its related text, whereby the present invention is described through given examples and provided embodiments for a better understanding of the invention, wherein:

FIG. 1 is a schematic diagram of a four-electrode biosensor according to an embodiment of the invention;

FIG. 2 is an illustrative block diagram of an analyte monitoring system according to one embodiment of the present invention;

FIG. 3 is a schematic diagram illustrating connection of an amperometric biosensor to a potentiostat according to one embodiment of the present invention;

FIG. 4 is a schematic diagram illustrating connection of a selector and an auxiliary power source to an amperometric biosensor according to one embodiment of the present invention; and

FIGS. 5A-5D are circuit diagrams of an analyte monitoring system according to one embodiment of the invention.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The present invention provides systems and methods that allow physicians or other health care workers to monitor a patient using a biosensor, such as an electro-chemical biosensor comprising an eletrolytic cell. The electro-chemical biosensor may contain an enzyme capable of reacting with a substance in a fluid, such as blood glucose, to generate electrical signals. These signals are sent to processor, which calculates the amount of substance in the fluid, for example, the blood glucose concentration in blood. The results can then be conveniently displayed for the attending physician. The device may also be specially designed to isolate the biosensor signals from interfering noise and electrical static, so that more accurate measurements can be taken and displayed. In some embodiments, the biosensor can operate continually when it is installed in the blood vessel, the results may be seen in real time whenever they are needed. This has the advantage of eliminating costly delays that occur using the old method of extracting blood samples and sending them off for laboratory analysis. In some instance, the biosensor is fitted to a catheter, such that it may be placed into the patient's blood stream. In this instance, use of the intravenous biosensor means that the patient does not suffer any discomfort from periodic blood drawing, or experience any blood loss whenever a measurement needs to be taken.

It must be understood that the systems and methods of the present invention may be used with any biosensor requiring continuous or substantially continuous biasing. For example, the systems and methods may be used with electro-chemical biosensors having eletrolytic cells, such as amperometric and potentiometric biosensors containing one or more electrodes used to measure an anlayte in a substance, such as glucose in blood, where the electrodes of the electrolytic cell require biasing to create a steady state mode for proper operation.

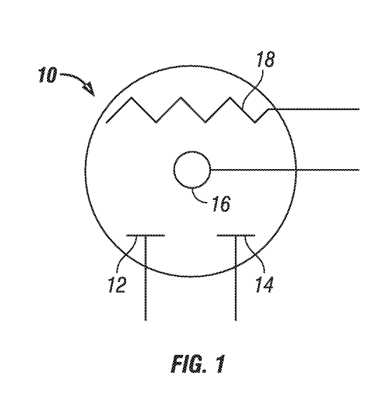

For example, FIG. 1 is a schematic diagram of an amperometric, four-electrode biosensor 10 which can be used in conjunction with the present invention. In the illustrated embodiment, the biosensor 10 includes two working electrodes: a first working electrode 12 and a second working electrode 14. The first working electrode 12 may be a platinum based enzyme electrode, i.e. an electrode containing or immobilizing an enzyme layer. In one embodiment, the first working electrode 12 may immobilize an oxidase enzyme, such as in the sensor disclosed in U.S. Pat. No. 5,352,348, the contents of which are hereby incorporated by reference. In some embodiments, the biosensor is a glucose sensor, in which case the first working electrode 12 may immobilize a glucose oxidase enzyme. The first working electrode 12 may be formed using platinum, or a combination of platinum and graphite materials. The second working electrode 14 may be identical in all respects to the first working electrode 12, except that it may not contain an enzyme Layer. The biosensor 10 further includes a reference electrode 16 and a counter electrode 18. The reference electrode 16 establishes a fixed potential from which the potential of the counter electrode 18 and the working electrodes 12 and 14 may be established. The counter electrode 18 provides a working area for conducting the majority of electrons produced from the oxidation chemistry back to the blood solution. Otherwise, excessive current may pass through the reference electrode 16 and reduce its service life.

The amperometric biosensor 10 operates according to an amperometric measurement principle, where the working electrode 12 is held at a positive potential relative to the reference electrode 16. In one embodiment of a glucose monitoring system, the positive potential is sufficient to sustain an oxidation reaction of hydrogen peroxide, which is the result of glucose reaction with glucose oxidase. Thus, the working electrode 12 may function as an anode, collecting electrons produced at its surface that result from the oxidation reaction. The collected electrons flow into the working electrode 12 as an electrical current. In one embodiment with the working electrode 12 coated with glucose oxidase, the oxidation of glucose produces a hydrogen peroxide molecule for every molecule of glucose when the working electrode 12 is held at a potential between about +450 mV and about +650 mV. The hydrogen peroxide produced oxidizes at the surface of the working electrode 12 according to the equation:

H₂O₂→2H⁺+O₂+2e⁻

The equation indicates that two electrons are produced for every hydrogen peroxide molecule oxidized. Thus, under certain conditions, the amount of electrical current may be proportional to the hydrogen peroxide concentration. Since one hydrogen peroxide molecule is produced for every glucose molecule oxidized at the working electrode 12, a linear relationship exists between the blood glucose concentration and the resulting electrical current. The embodiment described above demonstrates how the working electrode 12 may operate by promoting anodic oxidation of hydrogen peroxide at its surface. Other embodiments are possible, however, wherein the working electrode 12 may be held at a negative potential. In this case, the electrical current produced at the working electrode 12 may result from the reduction of oxygen. The following article provides additional information on electronic sensing theory for amperometric glucose biosensors: J. Wang, “Glucose Biosensors: 40 Years of Advances and Challenges,” Electroanalysis, Vol. 13, No. 12, pp. 983-988 (2001).

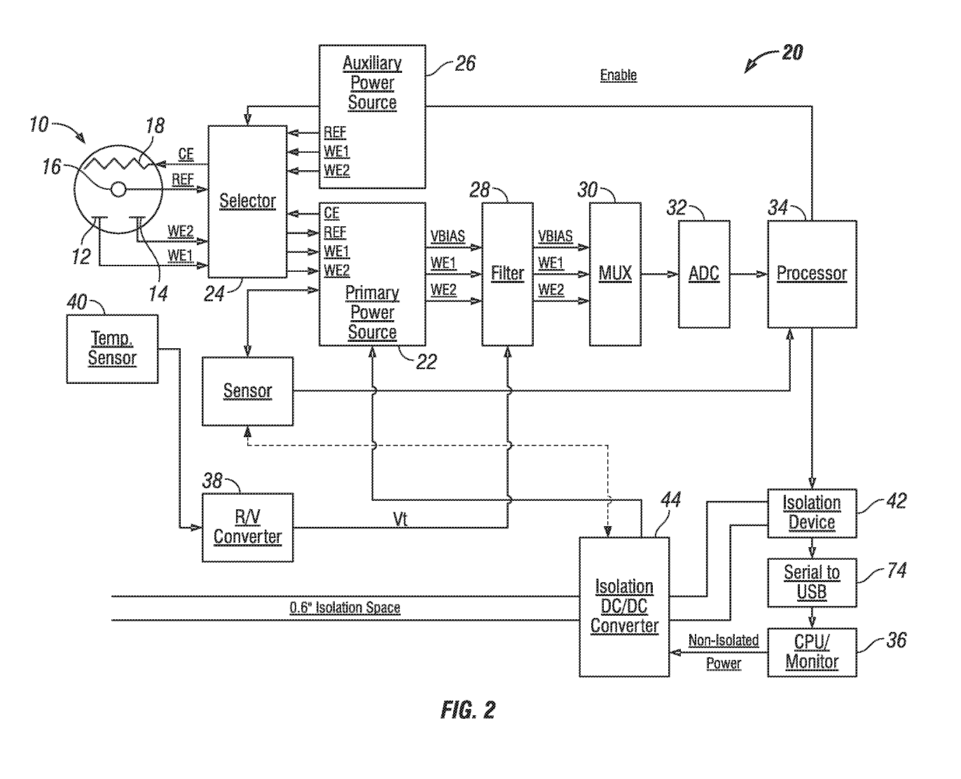

FIG. 2 illustrates a schematic block diagram of a system 20 for operating an electro-chemical biosensor such as an amperometric or potentiometric sensor, such as a glucose sensor. In particular, FIG. 2 discloses a system comprising an amperometric biosensor, such as the one described in FIG. 2. As more fully disclosed in U.S. patent application Ser. No. 11/696,675, filed Apr. 4, 2007, and titled Isolated Intravenous Analyte Monitoring System, a typical system for operating an amperometric sensor includes a potentiostat 22 in communication with the sensor 10. In normal operation, the potentiostat both biases the electrodes of the sensor and provides outputs regarding operation of the sensor. As illustrated in FIG. 2, the potentiostat 22 receives signals WE1, WE2, and REF respectively from the first working electrode 12, second working electrode 14, and the reference electrode 16. The potentiostat further provides a bias voltage CE input to the counter electrode 18. The potentiostat 22, in turn, outputs the signals WE1, WE2 from the working electrodes 12 and 14 and a signal representing the voltage potential VBIAS between the counter electrode 18 and the reference electrode 16.

A potentiostat is a controller and measuring device that, in an electrolytic cell, keeps the potential of the working electrode 12 at a constant level with respect to the reference electrode 16. It consists of an electric circuit which controls the potential across the cell by sensing changes in its electrical resistance and varying accordingly the electric current supplied to the system: a higher resistance will result in a decreased current, while a lower resistance will result in an increased current, in order to keep the voltage constant.

Another function of the potentiostat is receiving electrical current signals from the working electrodes 12 and 14 for output to a controller. As the potentiostat 22 works to maintain a constant voltage for the working electrodes 12 and 14, current flow through the working electrodes 12 and 14 may change. The current signals indicate the presence of an analyte of interest in blood. In addition, the potentiostat 22 holds the counter electrode 18 at a voltage level with respect to the reference electrode 16 to provide a return path for the electrical current to the bloodstream, such that the returning current balances the sum of currents drawn in the working electrodes 12 and 14.

While a potentiostat is disclosed herein as the first or primary power source for the electrolytic cell and data acquisition device, it must be understood that other devices for performing the same functions may be employed in the system and a potentiostat is only one example. For example, an amperostat, sometimes referred to as a galvanostat, could be used.

As illustrated in FIG. 2, the output of the potentiostat 22 is typically provided to a filter 28, which removes at least some of the spurious signal noise caused by either the electronics of the sensor or control circuit and/or external environmental noise. The filter 28 is typically a low pass filter, but can be any type of filter to achieve desired noise reduction.

In addition to electrical signal noise, the system may also correct analyte readings from the sensor based on operating temperature of the sensor. With reference to FIG. 2, a temperature sensor 40 may be collocated with the biosensor 10. Since chemical reaction rates (including the rate of glucose oxidation) are typically affected by temperature, the temperature sensor 40 may be used to monitor the temperature in the same environment where the working electrodes 12 and 14 of the biosensor are located. In the illustrated embodiment, the temperature sensor may be a thermistor, resistance temperature detector (RTD), or similar device that changes resistance based on temperature. An R/V converter 38 may be provided to convert the change in resistance to a voltage signal Vt that can be read by a processor 34. The voltage signal Vt represents the approximate temperature of the biosensor 10. The voltage signal Vt may then be output to the filter 28 and used for temperature compensation.

As illustrated in FIG. 2, a multiplexer may be employed to transfer the signals from the potentiostat 22, namely 1) the signals WE1, WE2 from the working electrodes 12 and 14; 2) the bias signal VBIAS representing the voltage potential between the counter electrode 18 and the reference electrode 16; and 3) the temperature signal Vt from the temperature sensor 40 to the processor 34. The signals are also provided to an analog to digital converter (ADC) 32 to digitize the signals prior to input to the processor.

The processor uses algorithms in the form of either computer program code where the processor is a microprocessor or transistor circuit networks where the processor is an ASIC or other specialized processing device to determine the amount of analyte in a substance, such as the amount of glucose in blood. The results determined by the processor may be provided to a monitor or other display device 36. As illustrated in FIG. 2 and more fully described in U.S. Patent App????, filed??, and titled Isolated Intravenous Analyte Monitoring System, the system may employ various devices to isolate the biosensor 10 and associated electronics from environmental noise. For example, the system may include an isolation device 42, such as an optical transmitter for transmitting signals from the processor to the monitor to avoid backfeed of electrical noise from the monitor to the biosensor and its associated circuitry. Additionally, an isolated main power supply 44 for supplying power to the circuit, such as an isolation DC/DC converter.

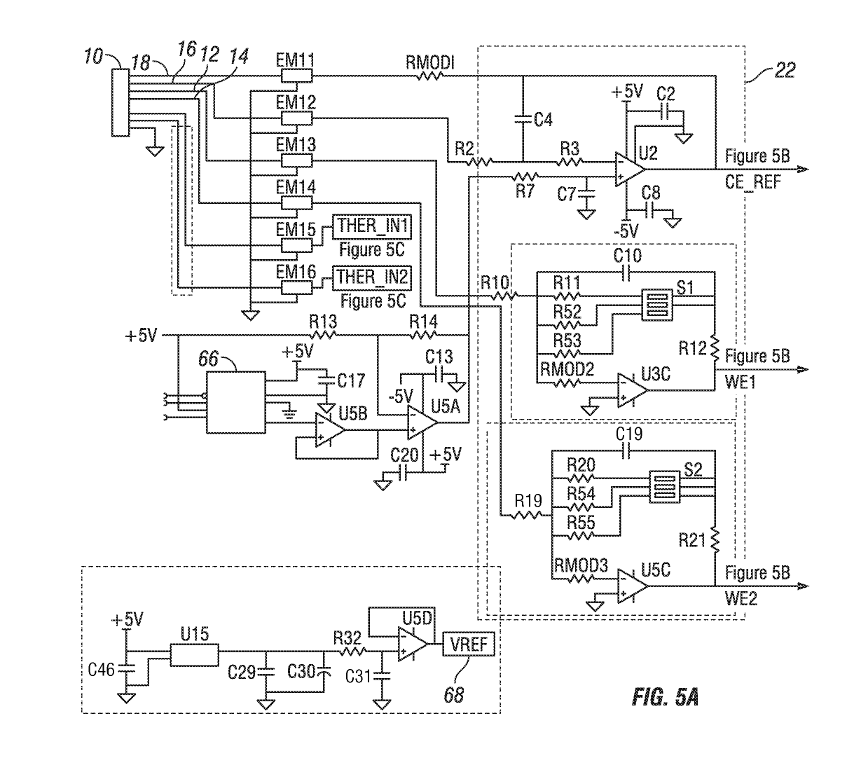

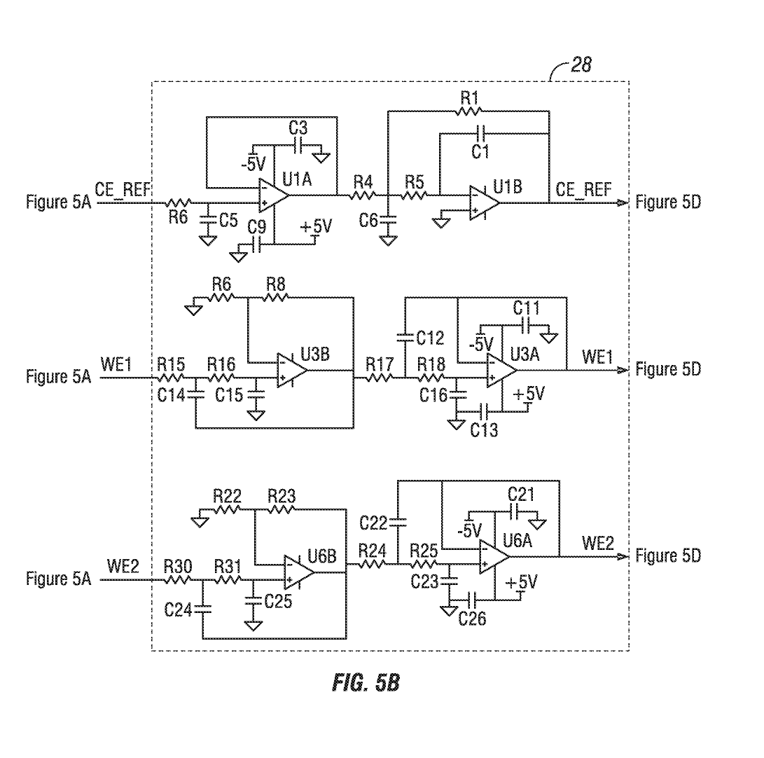

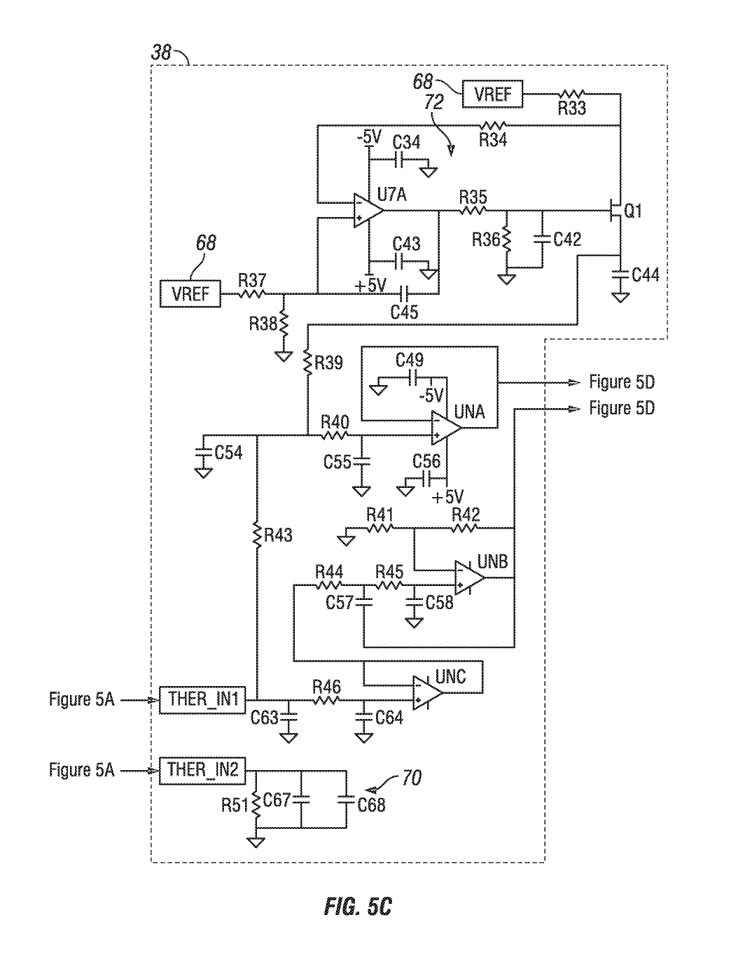

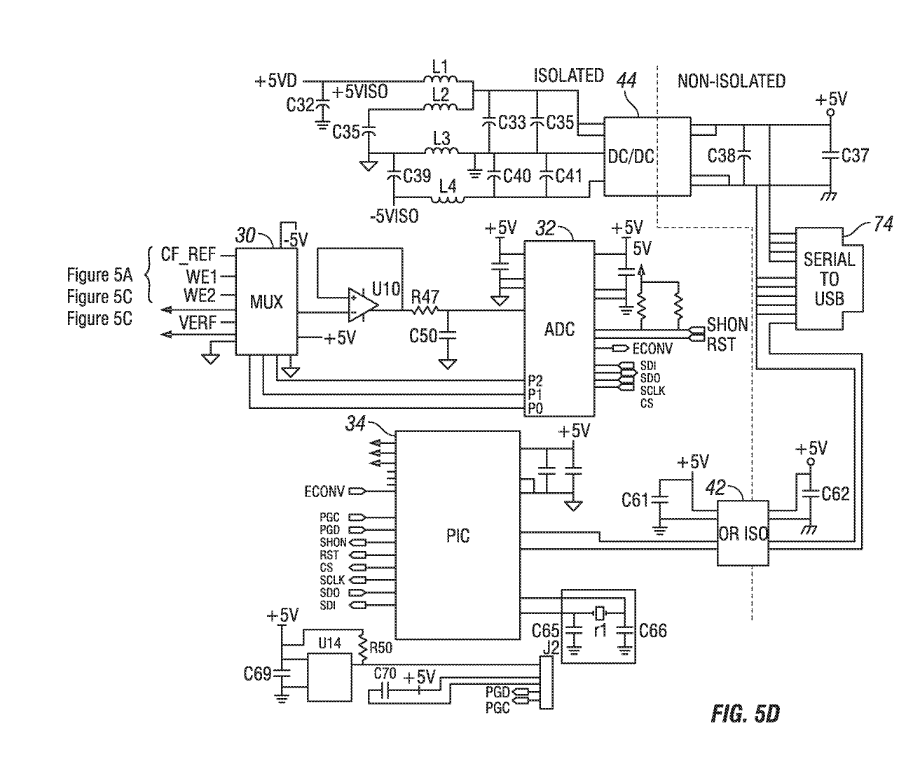

While FIG. 2 discloses a block diagram of a biosensor and circuit configuration, FIGS. 5A-5D discussed later below provide added details regarding circuit configuration.

As discussed previously, for proper operation of an electro-chemical biosensor, the electrodes of it electrolytic cell should remain biased to maintain a steady state or chemistry cell alignment. Disruption of bias voltage to the electrodes will result in a loss of steady state for the cell. Realignment of the cell may require an unacceptable run-in time, typically ranging from 15 minutes to over one (1) hour. For example, if the main power source 44 was temporarily disabled, such as in a power outage or disconnected such that the patient could be transported, the bio sensor may lose alignment due to loss of bias voltages. In light of this, the present invention provides systems and methods for sensing loss of power to the biosensor and application of auxiliary power to maintain bias voltages to the electrolytic cell of the biosensor, so as to prevent disruption of the operation of biosensor or at least minimize run-in time for realignment.

For example, as illustrated in the FIG. 2, the system 20 may further include a second or auxiliary power source 26. The auxiliary power source 26 is adapted for connection to the electrolytic cell of the biosensor 10. In this embodiment, the system includes a selector 24 located between the bio sensor 10 and the potentiostat 22 or other type of primary power source. The selector 24 is configured so as to connect either the potentiostat 22 or the auxiliary power source 26 to the electrolytic cell of the biosensor 10.

The selector 24 may take many forms depending on the embodiment. For example, in some embodiments, the selector may be a relay, such as single throw double pole relay. By activating or deactivating the relay, either the potentiostat 22 or the auxillary power source 26 can be connected to the biosensor 10. Other embodiments may employ transistor networks that operate as a relay. A processor, multiplexer, or other type of device may be deployed for alternatively connecting either the potentiostat or auxiliary power source to the biosensor. In short, any device capable of connecting either the potentiostat (or other primary power source) or auxiliary power source to the biosensor is contemplated.

In some embodiments, the selector may comprise a manual switch. In this embodiment, the patient's caretaker may toggle the selector to place the auxiliary power source in connection with the biosensor prior to disconnecting either the potentiostat 22 or main power supply 44 from the biosensor 10. In this way, the caretaker can ensure that the electrolytic cell of the biosensor is maintained in a steady state, while either the patient is being transported, or the biosensor is disconnected from the potentiostat or main power source for other reasons, or there is a power outage. In this embodiment, the selector may also be considered a sensor as detailed herein, as the selector essentially detects or indicates that the power from the potentiostat or main power supply is being removed from the biosensor.

With regard to FIG. 2, the system 22 may further include a sensor 50 for determining operation of either the potentiostat 22 or the main power supply 44. The sensor can be any type sensor. For example, it can be a voltage, current, inductive, capacitance, Fall Effect or similar type sensor connected to the outputs of either the potentiostat 22 or the main power supply 44. The sensor is either directly connected to the selector 24 or alternatively to the processor 34. In the embodiment illustrated in FIG. 2, the sensor is connected to the bias voltage output of the potentiostat, which is provided to the electrolytic cell of the biosensor 10. The sensor 50 is also connected to the processor 34. If the sensor 50 fails to detect a bias signal from the potentiostat, the processor 34 controls the selector 24 to connect the auxiliary power source 26 to the biosensor. When the sensor 50 indicates that potentiostat has a bias output, the processor controls the selector to disconnect the auxiliary power source 26 from the biosensor 10 and connect the potentiostat 22 to the biosensor.

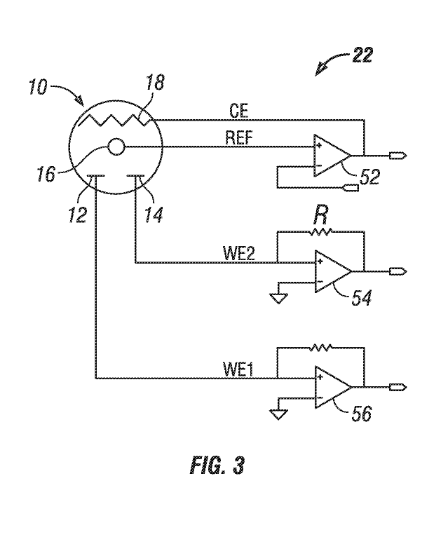

As discussed previously, the type and placement of the sensor can vary and FIG. 3 is only one exemplary embodiment of the present invention. The sensor can be connected to either the output of the potentiostat or the main power supply or it could be a simple push button operated manually by a caretaker or in some instances, the selector may act as the sensor by allowing a caretaker to manually toggle the switch.

As known in the art, some power sources have power down modes that are initiated when the power source is turned off. For example, the main power source 44 or the primary power source or potentiostat 22 may have a power down mode. In this instance, the sensor 50 could be associated with the power down mode with one or both of these power sources and detect when the power source enters a power down mode. The sensor 50 would then alert either the selector 24 or the processor 34 to connect the auxiliary power source 26.

FIG. 3 is an illustration of a typical potentiostat 22 as it would be connected to the biosensor 10. As illustrated, the potentiostat comprises three operational amplifiers, 52, 54, and 56. Operational amplifiers 54 and 56 are respectively coupled to working electrodes 12 and 14 of the biosensor 10 are referenced to ground. The other operational amplifier 52 is connected to both the reference 16 and the counter 18 electrodes. In this configuration, the operational amplifier 52 provides a bias voltage to the counter electrode 18. In the event of power loss from the potentiostat 22, the auxiliary power source is configured to replace the potentiostat in terms of providing bias signals to the electrodes of the sensor.

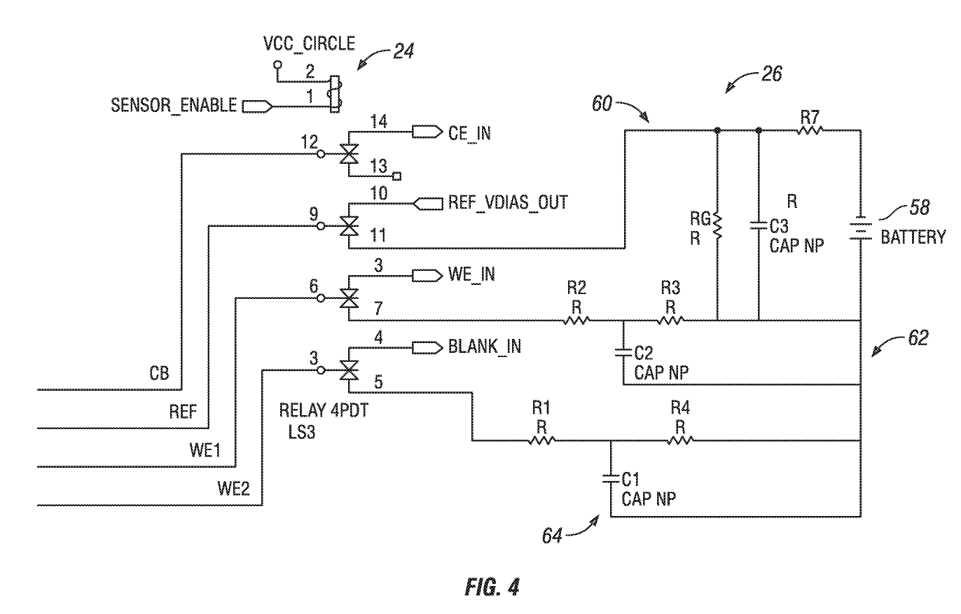

In this regard, FIG. 4 illustrates an embodiment of the auxiliary power source 26 in combination with a selector 24. The auxiliary power source of this embodiment comprises a power source 58, such as a battery or uninterruptible power source. The auxiliary power source 26 further includes three separate circuit paths 60-64 for connecting respectively to the reference electrode 16 and the first and second work electrodes 12 and 14. The circuit paths provide bias voltage or current to the electrodes. They each employ resistor/capacitor networks to tailor the voltage or current applied to the electrodes. For example, in one embodiment, bias voltages levels are provided to the electrodes so as to maintain a voltage level for each working electrode 12 and 14 of between about +450 mV and about +650 mV with respect to the reference electrode 16. In some embodiments, the auxiliary power source provides the same voltage to one or more electrodes and in other embodiments, different voltages are provided to some of the electrodes. The Alkaline 3.0 VDC battery is used to backup the sensor voltage potential of 0.700 VDC. The battery voltage is divided by two ratiometric resistors 2.49 Meg, and 750 K to provide voltage potential approximate 695 mv. Capacitor 1 uf is used as a energy holder voltage potential switch from internal voltage to battery bias. Additional three resistors of 20 Meg acting as a current limit to sensor for patient safety limit.

In the embodiment of FIG. 4, the selector 24 is a relay switch. In the disabled mode, the selector connects the potentiostat 22, not shown, to the biosensor 10 electrodes. When enabled, the selector disconnects the potentiostat 22 from the biosensor 10 and connects the outputs of the auxiliary power source 26 thereto. By toggling the relay, either the potentiostat or the auxiliary power source can be connected to the biosensor 10. The enable command for the selector 24 can either come directly from a sensor 50 or via a processor 34 in communication with both the sensor 50 and the selector 24 as illustrated in FIG. 2.

In addition to the disclosed systems, the present invention also discloses methods for maintaining bias signals to a biosensor. For example, in one embodiment, the method may comprise providing an electro-chemical biosensor capable of sensing an analyte concentration and outputting a signal corresponding to the analyte concentration. The method selectively couples either a first or a second power source to the biosensor based on whether one of the power sources is supplying power, so as to maintain the biosensor in a biased state. For example, the method couples the first power source to the biosensor if the first power source is outputting a signal to the sensor and couples the second power source to the biosensor if the first power is not outputting a signal to the sensor.

The above discussion describes the addition of an auxiliary power source, selector, and power outage sensor to an analyte monitoring system. It also provides exemplary circuit diagrams for these added elements to the system. Following is a discussion of exemplary circuit diagrams for a basic analyte monitoring system that includes added signal isolation.

With reference to FIG. 5A, the biosensor 10 is shown in the upper left, coupled to the potentiostat 22 via inputs EM11 through EM16. The signal lines to inputs EM11, EM12, EM13 and EM14 connect to the counter electrode 18, the reference electrode 16, the working electrode 12, and the working electrode 14, respectively as shown. The signal line to input EM15 connects to a first output from a thermistor 40, and the signal line to input EM16 connects to a second output from the thermistor 40. For convenience, the thermistor 40 outputs axe shown originating from a sensor block 10, which in this figure represents a local connection point. For example, the thermistor 40 may be integrated with or installed adjacent to the biosensor 10 in an intravenous catheter, in which case it may be convenient to terminate the thermistor 40 and sensor leads at the same connector. In another embodiment, the thermistor 40 and sensor leads may be terminated at separate locations.

The potentiostat 22 may include a control amplifier U2, such as an OPA129 by Texas Instruments, Inc., for sensing voltage at reference electrode 16 through input EM12. The control amplifier U2 may have low noise (about 15 nV/sqrt(Hz) at 10 kHz), an offset (about 5 μV max), an offset drift (about 0.04 μV max) and a low input bias current (about 20 fA max). The control amplifier U2 may provide electrical current to the counter electrode 18 to balance the current drawn by the working electrodes 12 and 14. The inverting input of the control amplifier U2 may be connected to the reference electrode 16 and preferably may not draw any significant current from the reference electrode 16. In one embodiment, the counter electrode 18 may be held at a potential of between about −600 mV and about −800 mV with respect to the reference electrode 16. The control amplifier U2 should preferably output enough voltage swing to drive the counter electrode 18 to the desired potential and pass current demanded by the biosensor 10. The potentiostat 22 may rely on R2, R3 and C4 for circuit stability and noise reduction, although for certain operational amplifiers, the capacitor C4 may not be needed. A resistor RMOD1 may be coupled between the counter electrode 18 and the output of the control amplifier U2 for division of return current through the counter electrode 18.

The potentiostat 22 may further include two current-to-voltage (I/V) measuring circuits for transmission and control of the output signals from the working electrode 12 and the working electrode 14, through inputs EM12 and EM13, respectively. Each I/V measuring circuit operates similarly, and may include a single stage operational amplifier U3C or U6C, such as a type TLC2264. The operational amplifier U3C or U6C may be employed in a transimpedance configuration. In the U3C measuring circuit, the current sensed by the working electrode 12 is reflected across the feedback resistors R11, R52 and R53. In the U6C measuring circuit, the current sensed in the working electrode 14 is reflected across the feedback resistors R20, R54 and R55. The operational amplifier U3C or U6C may generate an output voltage relative to virtual ground. The input offset voltage of the operational amplifier U3C or U6C adds to the sensor bias voltage, such that the input offset of the operational amplifier U3C or U6C may be kept to a minimum.

The I/V measuring circuits for the working electrode 12 and the working electrode 14 may also use load resistors R10 and R19 in series with the inverting inputs of operational amplifiers U3C and U6C, respectively. The resistance of the load resistors R10 and R19 may be selected to achieve a compromise between response time and noise rejection. Since the I/V measuring circuit affects both the RMS noise and the response time, the response time increases linearly with an increasing value of the load resistors R10 and R19, while noise decreases rapidly with increasing resistance. In one embodiment, each of load resistors R10 and R19 may have a resistance of about 100 ohms. In addition to the load resistors R10 and R19, the I/V amplifiers may also include capacitors C10 and C19 to reduce high frequency noise.

In addition, the I/V amplifiers of the potentiostat 22 may each include a Dual In-line Package (DIP) switch S1 or S2. Each DIP switch S1 and S2 may have hardware programmable gain selection. Switches S1 and S2 may be used to scale the input current from the working electrode 12 and the working electrode 14, respectively. For operational amplifier U3C, the gain is a function of RMOD2 and a selected parallel combination of one or more resistors R11, R52 and R53. For operational amplifier U6C, the gain is a function of RMOD3 and a selected parallel combination of one or more resistors R20, R54 and R55. Table 1 below illustrates exemplary voltage gains achievable using different configurations of switches S1 and S2.

TABLE 1
Exemplary Voltage Gain

| | | | I/V Output (U3C, | Voltage at A/D | |
|---|---|---|---|---|---|
| Switch Position (S1 and S2) | | | U6C) V per nA | Input | |
| OPEN | OPEN | OPEN | +4.9 V | +4.9 | V |
| OPEN | OPEN | CLOSED | 10 mV (1-20 nA | 200 | mV |
| | | | Scale) | | |
| OPEN | CLOSED | OPEN | 6.65 mV (1-30 nA | 133 | mV |
| | | | Scale) | | |
| CLOSED | OPEN | OPEN | 5 mV (1-40 nA | 100 | mV |
| | | | Scale) | | |

As shown from Table 1, three gain scale settings may be achieved, in addition to the fill scale setting. These settings may be selected to correspond to input ratings at the ADC 32.

The potentiostat 22, or a circuit coupled to the potentiostat 22, may further include a digital-to-analog converter (DAC) 66 that enables a programmer to select, via digital input, a bias voltage V_{BIAS} between the reference electrode 16 and the counter electrode 18. The analog output from the DAC 66 may be cascaded through a buffering amplifier U5B and provided to the non-inverting input of the amplifier U5A. In one embodiment, the amplifier U5A may be a type TLC2264 operational amplifier. The output of the amplifier USA may be bipolar, between ±5 VDC, to establish the programmable bias voltage V_{BIAS} for the biosensor 10. The bias voltage V_{BIAS} is the voltage between the counter electrode 18 and the reference electrode 16. Resistors R13 and R14 may be selected to establish a desired gain for the amplifier USA and the capacitors C13, C17 and C20 may be selected for noise filtration.

The potentiostat 22, or a circuit coupled to the potentiostat 22, may also establish a reference voltage 68 (VREF) for use elsewhere in the control circuits of the continuous glucose monitoring system 20. In one embodiment, the VREF 68 may be established using a voltage reference device U15, which may be an integrated circuit such as an Analog Devices type AD580M. In another embodiment, the reference voltage 68 may be established at about ±2.5 VDC. The reference voltage 68 may be buffered and filtered by an amplifier U5D in combination with resistors and capacitors R32, C29, C30 and C31. In one embodiment, the amplifier U5D may be a type TLC2264 device.

With reference now to FIG. 5B, the low-pass filter 28 is now described. The low-pass filter 28 may provide a two-stage amplifier circuit for each signal CE-REF, WE1 and WE2 received from the potentiostat 22. In one embodiment, a 1 Hz Bessel multi-pole low-pass filter may be provided for each signal. For example, the output signal CE_REF of amplifier U2 may be cascaded with a first stage amplifier U1A and a second stage amplifier U1B. The amplifier U1A, in combination with resistor R6 and capacitor C5, may provide one or more poles. One or more additional poles may be formed using an amplifier U1B in combination with R1, R4, R5, C1 and C6. Capacitors such as C3 and C9 may be added, as necessary, for filtering noise from the +/−5 VDC power supply. Similar low-pass filters may be provided for signals WE1 and WE2. For example, the amplifier U3B may be cascaded with an amplifier U3A to filter WE1. The amplifier U3B in combination with components such as R8, R9, R15, R16, C14 and C15 may provide one or more poles, and the amplifier U3A in combination with components such as R17, R18, C11, C12, C16 and C18 may provide one or more additional poles. Similarly, the amplifier U6B may be cascaded with an amplifier U6A to filter WE2. The amplifier U6B in combination with components such as R22, R23, R30, R31, C24 and C25 may provide a first pole, and the amplifier U6A in combination with components such as R24, R25, C21, C22 and C23 may provide one or more additional poles. Additional similar filters (not shown) may be added for filtering signal Vt received from the R/V converter 38. After the low-pass filter 28 filters out high-frequency noise, it may pass signals CE_REF, WE1 and WE2 to a multiplexer 30.

With reference to FIG. 5C, a temperature sensing circuit including the temperature sensor 40 and the RN converter 38 is now described. The R/V converter 38 receives input from the temperature sensor 40 at terminals THER_IN1 and THER_IN2. These two terminals correspond respectively to the inputs EM15 and EM16 of FIG. 5A that are connected across the temperature sensor 40. In one embodiment, the temperature sensor 40 may be a thermocouple. In another embodiment, the temperature sensor 40 may be a device such as a thermistor or a resistance temperature detector (RTD), which has a temperature dependent resistance. Hereinafter, for purposes of illustration only, the monitoring system 20 will be described that employs a thermistor as the temperature sensor 40.

Since chemical reaction rates (including the rate of glucose oxidation) are typically affected by temperature, the temperature sensor 40 may be used to monitor the temperature in the same environment where the working electrodes 12 and 14 are located. In one embodiment, the monitoring system 20 may operate over a temperature range of between about 15° C. and about 45° C. For continuous monitoring in an intravenous application, the operating temperature range is expected to be within a few degrees of normal body temperature. A thermistor 40 should therefore be selected that may operate within such a desired range, and that may be sized for installation in close proximity to the biosensor 10. In one embodiment, the thermistor 40 may be installed in the same probe or catheter bearing the biosensor 10.

The thermistor 40 may be isolated to prevent interference from other sensors or devices that can affect its temperature reading. As shown in FIG. 5C, the isolation of the thermistor 40 may be accomplished by including in the RN converter 38 a low-pass filter 70 at input THER_IN2. In one embodiment, the low-pass filter 78 may include a simple R-C circuit coupling input THER_1N2 to signal ground. For example, the filter 78 may be formed by a resistor R51 in parallel with a capacitance, e.g. capacitors C67 and C68.

With the thermistor 40 installed in an intravenous location, its resistance changes as the body temperature of the patient changes. The R/V converter 38 may be provided to convert this change in resistance to the voltage signal Vt. Thus, the voltage signal Vt represents the temperature of the biosensor 10. The voltage signal Vt may then be output to the low-pass filter 28 and used for temperature compensation elsewhere in the monitoring system 20.

In one embodiment, the thermistor 40 may be selected having the following specifications:

$${R}_{th} = {R}_{o} {e}^{β [ \frac{1}{T} - \frac{1}{{T}_{o}} ]} \qquad ( 1 )$$

where,
Rₜₕ is the thermistor resistance at a temperature T;
Rₒ is the thermistor resistance at temperature Tₒ;
β=3500° K.+/−5%;
Tₒ=310.15° K.; and
T is the blood temperature in K.

The reference resistance Rₛ is selected to yield:

$$\frac{{R}_{th}}{{R}_{s}} = 1.4308 + / - 0.010507 \qquad ( 2 )$$

To determine the blood temperature of a patient, equation (1) may be rewritten as:

$$T = {T}_{o} \frac{β}{{T}_{o} ln ( \frac{{R}_{th}}{{R}_{o}} ) + β} \qquad ( 3 )$$

To compensate the output from the biosensor 10 according to temperature, the resistance R₀ of the thermistor 40 may be converted into a voltage signal Vt. To accomplish this, the R/V converter 38 may provide a current source 72 for running a fixed current through the thermistor 40. One embodiment of a circuit for the current source 72 is shown at the top of FIG. 5C, and includes device Q1 and all components to the right of Q1.

In one embodiment, the current source 72 may provide a desired current through Q1. In one embodiment, the source current through Q1 may be between about 5 μA and about 15 μA. Q1 may be a JFET such as a type SST201. To control the JFET, the output of an operational amplifier U7A may be provided to drive the gate of Q1. The voltage VREF may be divided, as necessary, to place a voltage of about +2 VDC at the non-inverting input of the amplifier U7A. For example, a voltage divider may be formed by the resistors R37 and R38 between VREF and the amplifier U7A. The amplifier U7A may be configured as an integrator, as shown, by including a capacitor C45 in a feedback path between the output and the non-inverting input, and the resistor R34 in a feedback path from the drain of Q1 to the inverting input, to maintain the drain voltage of Q1 at about +2V. Components such as R36, C34, C42, C43 and C44 may be included, as desired, for filtration and stability.

The resistor R33 placed between the drain of Q1 and the +2.5V VREF may be selected to establish the source current of Q1 at a desired value. In one embodiment, the source current may be maintained at about 9.8 μA for compliance with a medical device standard such as IEC 60601-1. In one embodiment the thermistor 40 is classified under that standard as a Type CF device (i.e. a device that comes into physical contact with the human heart), and has limits for electrical current leakage that are set at 10 μA for normal operating conditions, and that are set at 50 μA for a single fault condition. The selection of resistor R33 and other components that make up the current source 72 may therefore depend on the desired end use application of the monitoring system 20.

One or more voltage signals Vt may be derived from the thermistor 40 by placing one or more reference resistors R39 and R43 in series with the thermistor 40 to carry the source current of Q1. The voltage signals created by the flow of the source current of Q1 through this series resistance may be filtered for electromagnetic interference (EMI) using capacitors C54 and C63. The voltage signals may be further filtered with passive signal poles formed by R40 and C55, and by R46 and C64. In one embodiment, these poles may be established to provide a crossover frequency at approximately 30 Hz. These passive filters protect amplifiers U11A, U11B and U11C from electrostatic discharge (ESD).

In one embodiment, the amplifiers U11A, U11B and U11C may be type TLC2264 devices selected for low noise (12 nV/sqrtHz at frequency=1 Hz), an offset of about 5 uV max, an offset drift of about 0.04 μV max, and an input bias current of about 1 pA max. The amplifier U11A may form a low-pass filter, and transmit a thermistor reference voltage Vt1 at resistor R43. The amplifier U11B may also form a low-pass filter, and transmit a thermistor input voltage Vt2 at the thermistor 40 that represents a sensed temperature. In one embodiment, the amplifier U11A or U11B may function as a two-pole Butterworth filter having a −3 dB point at about 5.0 Hz+/−0.6 Hz for anti-aliasing. Components such as R41, R42, R44, R45, C49, C56, C57 and C58 may be configured for this purpose, The amplifier U11C may be provided as a buffer amplifier at the input of the amplifier U11B.

The first and second voltage signals Vt output from the R/V converter 38 may then be received by the low-pass filter 72 for additional conditioning. In one embodiment, the low-pass filter 70 may provide a four-pole 5 Hz Butterworth filter for signals Vt. The Butterworth filters may double as anti-aliasing filters to create the four-pole response with a −3 dB point at about 5.0 Hz, and have a gain of about 20 (i.e. 26 dB) to provide an output from about 100 mV to about 200 mV per 1.0 nA.

The signals from the biosensor 10 and the thermistor 40 filtered by the low-pass filter 70 may then be output to the multiplexer 30. As shown in FIG. 5D, the multiplexer 30 may receive the signals CE_REF, WE1, WE2, VREF, and the two Vt signals (Vt1 and Vt2), and provide them to the analog to digital converter 32. A buffer amplifier U11 may be provided in this transmission path, along with filtering components such as R47 and C50.

In one embodiment, the multiplexer 30 may be an 8-channel analog multiplexer, such as a Maxim monolithic CMOS type DG508A. The channel selection may be controlled by the processor 34 via the output bits P0, P1 and P2 of the ADC 32. Table 2 illustrates an exemplary channel selection for the multiplexer 30.

The ADC 32 converts analog signals to discrete digital data. The ADC 32 may have n output bits (e.g. P0-P2) used for selecting analog input signals at a 2ⁿ-channel multiplexer 30. In one embodiment, the ADC 32 may be a Maxim type MAX1133BCAP device having a bipolar input with 16 bits successive approximation, single +5V DC power supply and low power rating of about 40 mW at 200 kSPS. The ADC 32 may have an internal 4.096 V_{REF}, which can be used as a buffer. The ADC 32 may be compatible with Serial Peripheral Interface (SPI), Queued Serial Peripheral Interface (QSPI), Microwire or other serial data link. In one embodiment, the ADC 32 may have the following input channels: bias voltage output (CE_REF), working electrode 12 (WE1), working electrode 14 (WE2), DAC converter voltage (DAC_BIAS), thermistor reference voltage (Vt1), thermistor input voltage (Vt2), reference voltage (2.5 VREF), and analog ground (ISOGND).

TABLE 2
Exemplary Channel Selection for the Multiplexer

| P2 | P1 | P0 | Mux. Channel | Analog Inputs Description |
|---|---|---|---|---|
| 0 | 0 | 0 | 0 | Reference electrode 16 control voltage |
| 0 | 0 | 1 | 1 | Working Electrode 12 current to voltage |
| 0 | 1 | 0 | 2 | Working electrode 14 current to voltage |
| 0 | 1 | 1 | 3 | Control & Reference bias voltage |
| 1 | 0 | 0 | 4 | Thermistor Reference voltage Vt1 |
| 1 | 0 | 1 | 5 | Thermistor Input voltage Vt2 |
| 1 | 1 | 0 | 6 | 2.5 V_{REF} voltage |
| 1 | 1 | 1 | 7 | ISOGND voltage |

The digital data from the ADC 32 may be transmitted to the processor 34. The processor 34 may be a programmable microprocessor or microcontroller capable of downloading and executing the software for accurate calculation of analyte levels sensed by the biosensor 10. The processor 34 may be configured to receive the digital data and, by running one or more algorithms contained in integral memory, may compute the analyte (e.g. glucose) level in the blood based on one or more digital signals representing CE_REF, WE1, WE2, DAC_BIAS and 2.5 VREF. The processor 34 may also run a temperature correction algorithm based on one or more of the foregoing digital signals and/or digital signal Vt1 and/or Vt2. The processor 34 may derive a temperature-corrected value for the analyte level based on the results of the temperature correction algorithm. In one embodiment, the processor 34 may be a Microchip Technology type PIC18F2520 28-pin enhanced flash microcontroller, with 10-bit A/D and nano-Watt technology, 32 k×8 flash memory, 1536 bytes of SRAM data memory, and 256 bytes of EEPROM.

The input clock to the processor 34 may be provided by a crystal oscillator Y1 coupled to the clock input pins. In one embodiment, the oscillator Y1 may be a CTS Corp. oscillator rated at 4 MHz, 0.005% or +/−50 ppm. Y1 may be filtered using the capacitors C65 and C66. The processor 34 may further include an open drain output U14, for example, a Maxim type MAX6328UR device configured with a pull-up resistor R50 that provides system power up RESET input to the processor 34. In one embodiment, the pull-up resistor R50 may have a value of about 10 kΩ. The capacitors C69 and C70 may be sized appropriately for noise reduction.

In one embodiment, data transfer between the processor 34 and the ADC 32 may be enabled via pins SHDN, RST, ECONV, SDI, SDO, SCLK and CS, as shown. An electrical connector J2, such as an ICP model 5-pin connector, may be used to couple pins PGD and PGC of the processor 34 to drain output U14. The connector J2 may provide a path for downloading desired software into the integral memory, e.g. flash memory, of the processor 34.

The processor 34 may output its results to a monitor, such as a CPU 36 via an optical isolator 42 and the serial-to-USB port 74. The optical isolator 42 may use a short optical transmission path to transfer data signals between the processor 34 and the serial-to-USB converter 74, while keeping them electrically isolated. In one embodiment, the optical isolator 42 may be an Analog Devices model ADuM1201 dual channel digital isolator. The optical isolator 42 may include high speed CMOS and monolithic transformer technology for providing enhanced performance characteristics. The optical isolator 42 may provide an isolation of up to 6000 VDC for serial communication between the processor 34 and the serial-to-USB converter 74. The filter capacitors C61 and C62 may be added for additional noise reduction at the +5 VDC inputs. At the capacitor C61, the +5 VDC power may be provided by an isolated output from the DC/DC converter 44. At the capacitor C62, the +5 VDC power may be provided from a USB interface via the CPU 36. In addition to these features, an isolation space 51 may be established (e.g., on a circuit board containing the isolated electrical components) between about 0.3 inches and about 1.0 inches to provide physical separation to electrically and magnetically isolate circuit components on the “isolated” side of the optical isolator 46 from circuit components on the “non-isolated” side. The components segregated onto “isolated” and “non-isolated” sides are indicated by the dashed line on FIG. 5D. In one embodiment, the isolation space may be 0.6 inches.

Generally, an isolation device or isolation means prevents noise from outside the isolated side of the circuit from interfering with signals sensed or processed within the isolated side of the circuit. The noise may include any type of electrical, magnetic, radio frequency, or ground noise that may be induced or transmitted in the isolated side of the circuit. In one embodiment, the isolation device provides EMI isolation between the isolated sensing circuit used for sensing and signal processing, and the non-isolated computer circuit used for power supply and display. The isolation device may include one or more optical isolators 42, DC/DC converters 44, isolation spaces 51, and one or more of the many electronic filters or grounding schemes used throughout the monitoring system 20.

The serial-to-USB converter 74 may convert serial output received through the optical isolator 42 to a USB communication interface to facilitate coupling of output from the processor 34 to the CPU 36. In one embodiment, the serial-to-USB converter 74 may be an FTDI model DLP-USB232M UART interface module. The converted USB signals may then be transmitted to the CPU 36 via a USB port for storage, printing, or display. The serial-to-USB converter 74 may also provide a +5 VDC source that may be isolated by isolation DC/DC converter 44 for use by potentiostat 22 and other electronic components on the isolated side of the circuit.

The CPU 36 may be configured with software for displaying an analyte level in a desired graphical format on a display unit 36. The CPU 36 may be any commercial computer, such as a PC or other laptop or desktop computer running on a platform such as Windows, Unix or Linux. In one embodiment, the CPU 36 may be a ruggedized laptop computer. In another embodiment, the graphics displayed by the CPU 36 on the display unit 36 may show a numerical value representing real-time measurements, and also a historical trend, of the analyte of interest to best inform attendant health care professionals. The real-time measurements may be continuously or periodically updated. The historical trend may show changing analyte levels over time, for example, over one or more hours or days, for an analyte level such as blood glucose concentration.

The CPU 36 may provide power to the isolation DC/DC converter 44 and may also provide power to the display unit 36. The CPU 36 may receive power from a battery pack or a standard wall outlet (e.g. 120 VAC), and may include an internal AC/DC converter, battery charger, and similar power supply circuits. The isolation DC/DC converter 44 may receive DC power from the CPU 36 via a bus. In one embodiment, this DC power may be a +5 VDC, 500 WA, +/−5% source provided, for example, via an RS232/USB converter (not shown). The +5 VDC supply may be filtered at the non-isolated side of isolation DC/DC converter 44 using capacitors such as C37 and C38.

The isolation DC/DC converter 44 converts non-isolated +5 VDC power to an isolated +5 VDC source for output onto the bus labeled ISOLATED PWS OUT. In addition, the isolation DC/DC converter 44 may provide a physical isolation space for added immunity from electrical and magnetic noise. In one embodiment, the isolation space may be between about 0.3 inches and about 1.0 inches. In another embodiment, the isolation space may be 8 mm. The isolation DC/DC converter 44 may be a Transitronix model TVF05 D05K3 dual +/−5V output, 600 mA, regulated DC/DC converter with 6000 VDC isolation. The dual outputs +5V and −5V may be separated by a common terminal, and filtered using capacitors C33 and C36 between +5V and common, and capacitors C40 and C41 between −5V and common. Additional higher-order filtering may be provided to create multiple analog and digital 5V outputs, and to reduce any noise that may be generated on the isolated side of the circuit by digital switching of the components such as the ADC 32 and the processor 34. For example, the +5V and −5V outputs may be filtered by inductors L1, L2, L3 and L4 configured with the capacitors C32, C35 and C39. In the configuration shown, these components provide a +5V isolated supply (+5 VD) for digital components, a +/−5V isolated supply (+5 VISO and −5 VISO) for analog components, and an isolated signal ground for analog components.

In one embodiment, components of an analyte monitoring system may be mounted on one or more printed circuit boards contained within a box or Faraday cage. The components contained therein may include one or more potentiostats 22, R/V converters 38, low-pass filters 28, multiplexers 30, ADCs 32, processors 34, optical isolators 42, DC/DC converters 44, and associated isolated circuits and connectors. In another embodiment, the same board-mounted components may be housed within a chassis that may also contain serial-to-USB converter 74 and the CPU 36.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

That which is claimed:

1. An analyte monitoring system, comprising:
a biosensor capable of sensing an analyte concentration and outputting a signal indicative of the analyte concentration;
first and second power sources, each selectively couplable to said biosensor, wherein said first and second power sources are capable of providing one or more bias signals to said biosensor; and
a selector coupled to said first and second power sources, wherein said selector selectively electrically couples one of said first and second power sources to said biosensor.

2. A system according to claim 1 further comprising a sensor in communication with said selector, said sensor being capable of determining whether a bias signal is being supplied to the biosensor, and wherein said selector selectively electrically couples one of said first and second power sources to said biosensor based on an output of said sensor.

3. A system according to claim 2, wherein said sensor is capable of sensing one of a voltage or a current output from said first power source, wherein if said sensor senses that said first power source is not outputting a voltage or a current, said selector electrically couples said second power source to said biosensor.

4. A system according to claim 1, wherein said selector is a switch capable of being manipulated by an operator.

5. A system according to claim 1, wherein said biosensor comprises two or more electrodes, and wherein said second power source is configured so as to provide one or more bias signals to the two or more electrodes of said biosensor, wherein said second power source is configured so as to provide two or more bias signals to the two or more electrodes of said biosensor.

6. A system according to claim 1, wherein said biosensor comprises at least a reference electrode and a work electrode, wherein said second power source is configured so as to provide a bias signal to both said reference and work electrodes, and wherein said selector is capable of coupling said second power source to both of the reference and work electrodes.

7. A system according to claim 1, wherein:
said biosensor comprises at least a reference electrode and first and second work electrodes,
said second power source is configured so as to provide a bias signal to each of said reference and first and second work electrodes,
said selector is a relay having contacts connected to each of said reference and first and second work electrodes and to said second power source and is capable of coupling said second power source to each of said reference and first and second work electrodes.

8. A system according to claim 1, wherein:
said biosensor comprises one or more electrodes,
said first power source is a potentiostat for biasing one or more electrodes of said biosensor,
said second power source comprises voltage nodes for biasing one or more electrodes of said biosensor, and
said selector is a relay capable of selectively applying biasing from either of said first power source or said second power source to the one or more electrodes of said biosensor.

9. An analyte monitoring system, comprising:
a biosensor capable of sensing an analyte concentration and outputting a signal corresponding to the analyte concentration;
a potentiostat selectively couplable to said biosensor, wherein said potentiostat is capable of both providing one or more biasing signals to said biosensor and receiving one or more signals from said biosensor;
an auxiliary power source capable of providing one or more biasing signals to said biosensor; and
a selector coupled to said potentiostat and said auxiliary power source, wherein said selector selectively electrically couples one of said potentiostat and said auxiliary power source to said biosensor.

10. A system according to claim 9 further comprising a sensor in communication with said selector, said sensor being capable of determining whether a bias signal is being supplied to the biosensor, and wherein said selector selectively electrically couples one of said first and auxiliary power sources to said biosensor based on an output of said sensor.

11. A system according to claim 9, wherein said sensor is capable of sensing one of a voltage or a current output from said potentiostat, wherein if said sensor senses that said first power source is not outputting a voltage or a current, said selector electrically couples said auxiliary power source to said biosensor.

12. A system according to claim 9, wherein said sensor is a switch capable of being manipulated by an operator.

13. A system according to claim 9, wherein said biosensor comprises two or more electrodes, and wherein said auxiliary power source is configured so as to provide one or more bias signals to the two or more electrodes of said biosensor, wherein said auxiliary power source is configured so as to provide two or more bias signals to the two or more electrodes of said biosensor.

14. A system according to claim 9, wherein said biosensor comprises at least a reference electrode and a work electrode, wherein said auxiliary power source is configured so as to provide a bias signal to both the reference and work electrodes, and wherein said selector is capable of connecting said auxiliary power source to both of the reference and work electrodes.

15. A system according to claim 9, wherein:
said biosensor comprises at least a reference electrode and first and second work electrodes,
said auxiliary power source is configured so as to provide a bias signal to each of said reference and first and second work electrodes,
said selector is a relay having contacts connected to each of said reference and first and second work electrodes and to said auxiliary power source and is capable of coupling said auxiliary power source to each of said reference and first and second work electrodes.

16. A method controlling operation of a biosensor comprising:
providing a biosensor capable of sensing an analyte concentration and outputting a signal corresponding to the analyte concentration; and
selectively coupling either a first or a second power source to the biosensor based on whether one of said power sources is supplying a bias signal to the biosensor, so as to maintain the biosensor in a biased state.

17. A method according to claim 16 further comprising sensing operation of the first power source, and said coupling comprises coupling the first power source to the biosensor if the said sensing step senses that the first power source is outputting a signal and coupling the second power source to the biosensor if said sensing step senses that the first power source is not outputting a signal.

18. A method according to claim 16, wherein the biosensor comprises two or more electrodes, said method further comprising providing one or more bias signals via the second power source to the two or more electrodes of the biosensor, wherein said providing step provides two or more voltages via the second power source to the two or more electrodes of the biosensor.

19. An analyte monitoring system, comprising:
a electro-chemical biosensor comprising an electrolytic cell, wherein said electro-chemical biosensor is capable of sensing an analyte concentration and outputting a signal corresponding to the analyte concentration;
a first power source selectively couplable to said biosensor, wherein said first power source is capable of providing a biasing signal to the electrolytic cell of said biosensor;
an auxiliary power source capable of providing a biasing signal to the electrolytic cell of said biosensor; and
a selector coupled to said first power source and said auxiliary power source, wherein said selector selectively electrically couples one of said potentiostat and said auxiliary power source to said biosensor.

20. A system according to claim 19 further comprising a sensor in communication with said selector, said sensor being capable of determining whether a bias signal is being supplied to the biosensor, and wherein said selector selectively electrically couples one of said first and auxiliary power sources to said biosensor based on an output of said sensor.

21. A system according to claim 19, wherein said sensor is capable of sensing one of a voltage or a current output from said potentiostat.

22. A system according to claim 19, wherein said sensor is a switch capable of being manipulated by an operator.

23. An analyte monitoring system, comprising:
a electro-chemical biosensor comprising an electrolytic cell, wherein said electro-chemical biosensor is capable of sensing an analyte concentration and outputting a signal corresponding to the analyte concentration;
a first power source selectively couplable to said biosensor, wherein said first power source is capable of providing a biasing signal to the electrolytic cell of said biosensor;
an auxiliary power source capable of providing a biasing signal to the electrolytic cell of said biosensor;
a sensor capable of determining whether a bias signal is being supplied to said biosensor; and
a selector coupled to said first power source and said auxiliary power source, wherein said selector selectively electrically couples one of said potentiostat and said auxiliary power source to said biosensor based on an output of said sensor.
